(19) United States
(12) Patent Application Publication
(10) Pub. No.: US 2012/0295292 A1
(43) Pub. Date: Nov. 22, 2012

(54) Detecting Protein Arginine Deiminase (PAD) Activity in Human Tissues and Sera

(75) Inventors: Paul R. Thompson, Jupiter, FL (US); Lorne J. Hofseth, Columbia, SC (US); Bryan A. Knuckley, Jacksonville, FL (US)

(73) Assignee: University of South Carolina, Columbia, SC (US)

(21) Appl. No.: 13/476,379

(22) Filed: May 21, 2012

Related U.S. Application Data
(60) Provisional application No. 61/487,934, filed on May 19, 2011.

(51) Int. Cl. G01N 21/78; C12Q 1/34

(52) U.S. Cl. 435/18

(57) ABSTRACT

Methods for diagnosing disease that is characterized by an overabundance of citrullinated proteins such as colitis, rheumatoid arthritis, and/or malignant cancer in a subject are provided. The PAD protein activity level can be measured in a tissue sample (e.g., serum) of the subject and then compared to a control PAD protein activity range of a control group. A finding of an increased PAD protein activity level in the tissue sample of the subject compared to the PAD protein activity level of the control group is indicative of disease characterized by an overabundance of citrullinated proteins in the subject.

### PRIORITY INFORMATION

The present application claims priority to U.S. Provisional Patent Application Ser. No. 61/487,934 titled “Detecting Protein Arginine Deiminase (PAD) Activity in Human Tissues and Sera” of Thompson, et al. filed on May 19, 2011, the disclosure of which is incorporated by reference herein.

### GOVERNMENT SUPPORT CLAUSE

This invention was made with government support under NIH 5P20RRO17698-08 awarded by National Institutes of Health. The government has certain rights in the invention.

### BACKGROUND

The abundance of citrullinated proteins in human tissue has become a hallmark of many diseases, including cancer, rheumatoid arthritis, colitis, and multiple sclerosis. Numerous biochemical reports have suggested that the immune system recognizes these citrullinated proteins as foreign, causing a break in tolerance, and leading to the onset and progression of disease such as rheumatoid arthritis. Rheumatoid arthritis is an autoimmune disease affecting the synovium of the joints and has been shown to decrease life expectancy by roughly 5 to 10 years. In addition to their presence in rheumatoid arthritis, colitis, and cancer, overabundance of citrullinated proteins appears to play a role in the development and progression of many other diseases, e.g., Multiple Sclerosis, osteoarthritis, ankylosing spondylitis, Alzheimer's diseases, glaucoma, HIV/AIDS, and scrapie.

Proteins are citrullinated by a class of enzymes termed Protein Arginine Deiminases (PADs), which convert peptidyl-arginine to peptidyl-citrulline through an enzymatic reaction called deimination. PADs catalyze the post-translational modification of peptidyl-arginine to peptidyl-citrulline through a hydrolytic mechanism. This modification has alternatively been termed citrullination or deimination. In humans, there are five calcium-dependent PAD. isozymes denoted PADs 1, 2, 3, 4, and 6 (for historical reasons there is no PADS). These PAD isozymes are clustered on chromosome 1p35-36, and share approximately 59-71% sequence homology at the amino acid sequence level. PADs 1, 2, 3, and 6 are cytoplasmic enzymes, whereas PAD4 is most predominantly directed to the nucleus by a nuclear localization signal encoded in its N-terminus, Furthermore, the tissue-specific distribution varies between isozymes. For example, PAD4 is expressed in immune cells (i.e., lymphocytes, granulocytes, monocytes), as well as numerous cancer cell lines and tumors.

Overexpression of PAD4 has been identified in malignant tumors of breast, endometrial, colorectal, ovarian, lung, and uterine cancers, but only basal levels exist in benign tumors. More importantly, malignant cancer patients show an elevated expression of PAD4 protein in their blood, whereas the blood of patients with benign tumors or non-tumor inflammatory conditions has not exhibited increased PAD4 expression. Further support for the role of PAD4 in tumor progression has been strengthened by a study conducted by Chang et al., who found that the removal of a tumor significantly reduced PAD4 levels in the serum. Development of PAD inhibitors has become a focus of much research due to the apparent roles of the PADs in certain cancers. In recent years, a few PAD inhibitors have been described in the literature. Most notably, the haloacetamidine-based compounds, F- and Cl-amidine, are the most potent inhibitors described to date, and are described in U.S. Pat. No. 7,964,636 of Thompson, et al., which is incorporated by reference herein. These compounds irreversibly modify an active site Cys that is essential for catalysis. The efficacy of F- and Cl-amidine are currently being investigated in a series of murine disease models.

A need exists for tools and methods for early stage detection and diagnosis of disease states that are characterized by the overabundance of citrullinated proteins. More specifically, systems and methods that can be utilized to detect and diagnose such disease states through determination of dysregulated PAD activity in a subject would be of great benefit in the art.

### SUMMARY

Objects and advantages of the invention will be set forth in part in the following description, or may be obvious from the description, or may be learned through practice of the invention.

Methods are generally provided for diagnosing disease that is characterized by an overabundance of citrullinated proteins in a subject. Diseases that can be diagnosed by the disclosed methods and systems can include, for example, colitis, rheumatoid arthritis, and/or malignant cancer. According to the method, the PAD protein activity level can be obtained for an active PAD-containing tissue sample (e.g., serum) of the subject and then compared to a control PAD protein activity level as determined for the same tissue type. A finding of an increased PAD protein activity level in the tissue sample of the subject compared to the PAD protein activity level of the control group is indicative of a disease state in the subject. For instance, a finding that the PAD protein activity level in the tissue sample of the subject is greater than about twice the PAD protein activity level of the control group is indicative of colitis in the subject. In one embodiment, the method can be specifically targeted to measure the activity level of PAD4.

Also disclosed are assay systems as may be utilized for carrying out a diagnosis method. For example, an assay system can include a substrate for a PAD, and the PAD protein activity level in a sample of a subject can be measured via incubating the sample with the substrate. During the incubation, PAD in the sample can interact with the substrate to form a detectable reaction product. Detection of the reaction product can be utilized to determine the PAD activity level, which can then be compared to a control PAD activity level for non-affected individual for diagnosis of a disease that is characterized by the overabundance of citrullinated proteins.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIG. 1 shows an exemplary reaction of using PADs to catalyze the conversion of peptidyl-arginine to peptidyl-citrulline.

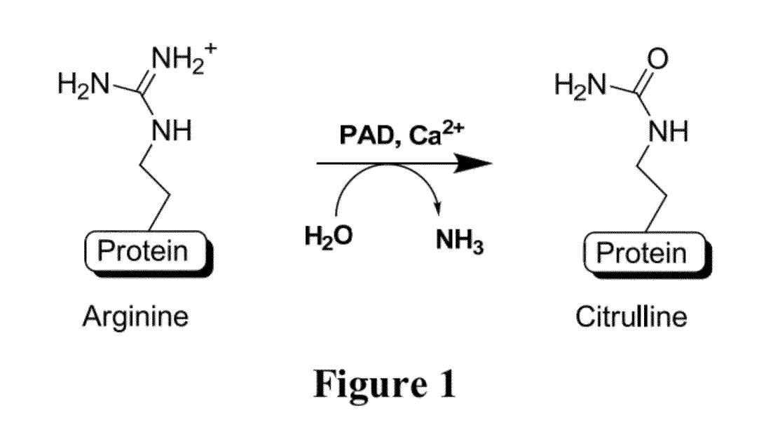

FIG. 2 shows PAD activity measured in the serum (A) and spleen (B) of mice, according to the examples testing three groups were tested: control, DSS (disease-induced), and DSS+Cl-amidine (disease induced in conjunction with PAD inhibition).

### Definitions

Throughout the specification, several terms are employed that are defined in the following paragraphs.

As used herein, the term “subject” refers to a human or another mammal (e.g., primate, dog, cat, goat, horse, pig, mouse, rat, rabbit, and the like), that can be afflicted with a disease, but may or may not have the disease. In many embodiments of the present invention, the subject is a human being. In such embodiments, the subject is often referred to as an “individual”. The term “individual” does not denote a particular age, and thus encompasses children, teenagers, and adults.

The term “biological sample” is used herein in its broadest sense. A biological sample is generally obtained from a subject. A sample may be of any biological tissue or fluid with which PAD activity levels may be assayed. Frequently, a sample will be a “clinical sample”, i.e., a sample derived from a patient. Such samples include, but are not limited to, bodily fluids which may or may not contain cells, e.g., blood (e.g., whole blood, serum or plasma), urine, synovial fluid, saliva, and joint fluid; tissue or fine needle biopsy samples, such as from bone or cartilage, and archival samples with known diagnosis, treatment and/or outcome history. Biological samples may also include sections of tissues such as frozen sections taken for histological purposes. The term “biological sample” also encompasses any material derived by processing a biological sample. Derived materials include, but are not limited to, cells (or their progeny) isolated from the sample or proteins extracted from the sample. Processing of a biological sample may involve one or more of: filtration, distillation, extraction, concentration, inactivation of interfering components, addition of reagents, and the like.

The terms “normal” and “healthy” are used herein interchangeably. They refer to a subject that has not shown any disease symptoms, and that has not been diagnosed with any disease that is characterized by an overabundance of citrullinated proteins. Preferably, a normal subject is not on medication affecting a disease and has not been diagnosed with any disease. In certain embodiments, normal subjects have similar sex, age, and/or body mass index as compared with the subject from which the biological sample to be tested was obtained. The term “normal” is also used herein to qualify a sample obtained from a healthy subject.

In the context of the present invention, the term “control”, when used to characterize a subject, refers to a subject that is healthy and has not been diagnosed with a specific disease. The term “control group” refers to a collection of samples taken from multiple control subjects.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. The term protein can also refer to sequences in either the active or inactive form. In certain embodiments, the amino acid sequence is a full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains such as oxidation of sulfhydryl groups. Thus, the term “protein” (or its equivalent terms) is intended to include the amino acid sequence of the full-length native protein, or a fragment thereof, subject to those modifications that do not significantly change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

### DETAILED DESCRIPTION OF INVENTION

The following description and other modifications and variations to the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the following description is by way of example only, and is not intended to limit the invention.

According to the present disclosure, methods and systems have been developed for diagnosing disease that is associated with an overabundance of citrullinated proteins. More specifically, it has been determined that the activity level of one or more PAD proteins can be utilized to diagnose such disease states. Accordingly, a simple and straightforward assay method and system is generally provided for detecting PAD activity in mammalian tissue. In one embodiment, the assay can allow for the detection of PAD activity in sera and observance of significant differences in the activity levels in normal tissue versus diseased tissue. This assay is particularly suitable for samples that naturally contain active PAD proteins, such as sera, thus creating a useful tool for early stage disease incidence and progression, as well as disease severity. The ability to rapidly screen for dysregulated PAD activity in human tissues and sera can provide for the early detection and diagnosis of diseases that are characterized with an overabundance of citrullinated proteins, such as malignant tumors (e.g., breast cancer, endometrial cancer, colorectal cancer, ovarian cancer, lung cancer, and uterine cancer), rheumatoid arthritis, colitis, multiple sclerosis, osteoarthritis, ankylosing spondylitis, Alzheimer's diseases, glaucoma, HIV/AIDS, and/or scrapie.

The ability to detect elevated levels of PAD protein activity in human serum or tissue can provide early diagnosis for PAD-related diseases. Herein, a facile and straightforward system is generally provided for measuring PAD activity in mammalian tissue. According to one embodiment, the system can utilize a solution based assay.

According to this embodiment, the PAD protein activity level can be determined for a tissue sample of the subject. In general, the sample can be obtained from tissue or other material that is known to contain a high level of the active form of the targeted PAD proteins. For example, the sample can be a blood or sera sample, as these materials contain a high level of the active PAD proteins.

The detection system can be a solution-based system that can provide the necessary substrates for the active PAD enzymes in the sample. Substrates for the PAD catalyzed citrullination reaction include an L-arginine containing compound and water, with the enzymatically catalyzed reaction products including the L-citrulline product and ammonia, as is known. The L-arginine containing substrate can be any compound that can provide the targeted L-arginine structure to the PAD enzyme, as is known. For instance, in one embodiment, the L-arginine substrate can be a proteinaceous compound such as a polypeptide including one or more L-arginine peptides in the chain. In another embodiment, the L-arginine substrate can include L-arginine as the only peptide in the compound. For example, the L-arginine substrate can be N-benzoyl-L-arginine ethyl ester, which includes L-arginine as the only peptide component, so as to prevent any undesired enzymatic reactions in the sample.

Following incubation of the sample with the substrate, the mixture can be examined to determine the PAD activity level of the sample. Incubation can be carried out for a period so as to ensure adequate interaction between the sample and the reagents, for instance for a period of time between about 5 minutes and several hours, e.g., between about 1 and about 5 hours. In general, PAD activity level can be determined by detection of a product of the PAD-catalyzed substrate reaction. For instance, PAD activity level can be determined by detection of the presence or quantity of one or both of the L-citrulline product or the ammonia product of the PAD enzyme catalyzed reaction.

By way of example, ammonia present in the incubated solution can be detected according to convention methods such as a color-developing method based on indophenol production, Nessler's method, phenosafaanin method, ninhydrin method, ammonia ion electrode method, measuring the change in optical density caused by the reaction of glutamic acid dehydrogenase with a-ketoglutarate in the presence of NADH or NADPH, etc. Satisfactory results can be obtained by employing any of these methods.

Following determination of the PAD activity level in the sample, the PAD activity level can be compared to a control PAD protein activity range of a control value. The control valued can be a value that has been determined for individuals that are not affected with the disease. It has been found that a finding of an increased PAD protein activity level in the tissue sample of the subject compared to the PAD protein activity level of the control group is indicative of disease in the subject that is associated with overabundance of citrullinated proteins. For example, a finding that the PAD protein activity level in the tissue sample of the subject is greater than about twice the PAD protein activity level of the control group can be particularly indicative of the PAD-related disease in the subject.

The present disclosure may be better understood with reference to the Example set forth below.

### EXAMPLE

It has been demonstrated that increased PAD activity can be detected in the sera and tissues of mice with DSS-induced colitis.

A mouse model of colitis was used to develop a method for detecting disease incidence and disease severity by measuring the PAD activity present in serum. In this model, mice were intravenously or orally administered 2% dextran sulfate sodium (DSS) for 1 week to induce colitis. This model induced moderate to severe colon inflammation, shortening of the colon, and ulcer formation. The efficacy was tested of the PAD inhibitor, Cl-amidine, to determine if PAD activity was diminished in a group of Cl-amidine treated mice versus a group receiving no treatment with the PAD inhibitor. As a control, mice were given water without 2% DSS.

To determine PAD activity levels within the mice, serum samples were collected, then resuspended in Homogenization Buffer (50 mM HEPES pH 7.6, 10% glycerol, 1% NP-40, 1 mM PMSF, and 2 mM DTT) and homogenized using a glass dounce homogenizer. Serum samples were then centrifuged at 10,600 g for 20 min to remove cellular debris. The protein concentration was measured using the Lowry assay. PAD activity was determined by incubating serum samples in a Reaction Buffer (100 mM 4-(2-hydroxyethyl)-1-piperazineethanesulfonic acid (“HEPES”) pH 7.6, 500 mM NaCl, mM dithiothreitol (“DTT”), 100 mM CaCl₂, and 100 mM N-benzoyl-L-arginine ethyl ester (“BAEE”)) as substrate for the PAD. Incubation was carried out for 2 h at 37° C. in duplicate before freezing in liquid nitrogen.

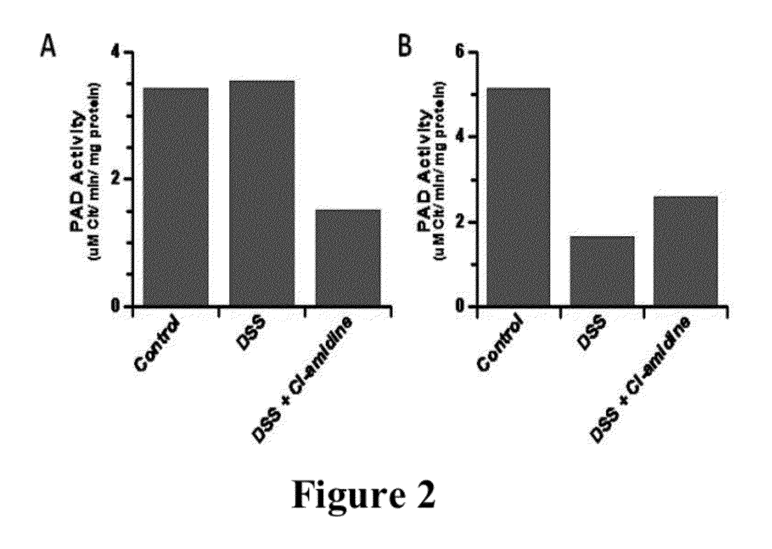

Following, 200 μL of COLDER (COLor Developing Reagent) for determination of ammonia was added, vortexed, and incubated at 95° C. for 30 min. Samples were aliquoted in a 96-well plate, the absorbance was measured at 540 nm, and compared to a standard curve of known citrulline concentrations. As a control, samples were added to the Reaction Buffer, frozen immediately in liquid nitrogen, and then baseline citrulline levels for each sample were subtracted from the total citrulline produced. As can be seen with reference to FIG. 2A, the mice treated with Cl-amidine showed a significantly (p>0.005) greater than 2.3-fold decrease in PAD activity as compared to the no treatment mice (FIG. 2). Similar results were obtained in tissues obtained from mouse colons (not shown).

For comparison, an identical protocol was carried out for samples obtained from spleen tissue. Spleen tissue is known to contain high levels of inactive form of the PAD protein, rather than the active form as is found in the sera. Results are shown in FIG. 2B. As can be seen, PAD activity levels were higher in the control mice as compared to the DSS-induced colitis mice (˜2.5-fold) and the DSS-induced/PAD inhibitor treated mice. This is believed to be due to the low concentration of active PAD enzyme in the spleen.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A method of diagnosing a disease characterized by an overabundance of citrullinated proteins in a subject, the method comprising:
measuring a PAD protein activity level in a tissue sample of the subject; and
comparing the PAD protein activity level in the tissue sample of the subject to a control PAD protein activity range for tissue of the same type of a control group;
wherein a finding of an increased PAD protein activity level in the tissue sample of the subject compared to the PAD protein activity level of the control group is indicative of the disease characterized by an overabundance of citrullinated proteins in the subject.

2. The method of claim 1, wherein the disease characterized by an overabundance of citrullinated proteins is colitis.

3. The method of claim 1, wherein the method determines an activity level of PAD4 protein.

4. The method of claim 1, wherein a finding that the PAD protein activity level in the tissue sample of the subject is at least about twice the PAD protein activity level of the control group is indicative of the disease characterized by an overabundance of citrullinated proteins in the subject.

5. The method of claim 1, wherein the tissue sample comprises serum.

6. The method of claim 1, wherein measuring the PAD protein activity level in the tissue sample of the subject comprises:
incubating the serum with a color developing reagent that is reactive with a PAD catalyzed reaction product in the sample; and
measuring the absorbance of the sample to determine the concentration of the PAD catalyzed reaction product in the sample.

7. The method of claim 1, wherein the diseased characterized by an overabundance of citrullinated proteins is rheumatoid arthritis.

8. The method of claim 1, wherein the disease characterized by an overabundance of citrullinated proteins is a malignant cancer.

9. The method of claim 8, wherein the malignant cancer is breast cancer, endometrial cancer, colorectal cancer, ovarian cancer, lung cancer, uterine cancer, or combinations thereof.

10. A system for diagnosing a disease characterized by an overabundance of citrullinated proteins in a subject, the system comprising:
a substrate for a PAD protein, the substrate comprising an L-arginine peptide,
a reagent for determining the presence or quantity of a product of a PAD-catalyzed reaction of the substrate; and
a control value for PAD activity level of a tissue sample as determined for individuals that are not affected with the disease characterized by an overabundance of citrullinated proteins in a subject.

11. The system of claim 10, wherein the reagent determines the presence or quantity of ammonia.

12. The system of claim 10, wherein the substrate is a polypeptide.

13. The system of claim 10, wherein the substrate contains L-arginine as the only peptide of the substrate.

14. The system of claim 13, wherein the substrate is N-benzoyl-L-arginine ethyl ester.
